(19) United States
(12) Patent Application Publication
(10) Pub. No.: US 2009/0176251 A1
(43) Pub. Date: Jul. 9, 2009

(54) Methods for Diagnosing Celiac Disease Based on the Level of Anti-Gliadin and Anti-tTG IgA and IgG Antibodies

(76) Inventors: Walter L. Binder, San Diego, CA (US); Donna L. Gustafson, San Diego, CA (US); Rufus Burlingame, San Diego, CA (US)

Correspondence Address: THE NATH LAW GROUP, 112 South West Street, Alexandria, VA, 22314, US

(21) Appl. No.: 11/971,127

(22) Filed: Jan. 8, 2008

(51) Int. Cl. G01N 33/53; G01N 33/00

(52) U.S. Cl. 435/7.9; 435/7.1; 435/7.92

(57) ABSTRACT

The present invention is in the field of diagnosing celiac disease. More particularly, the present invention relates to a method to diagnose celiac disease based on the level of anti-gliadin and anti-tissue transglutaminase antibodies.

### TECHNICAL FIELD

The present invention is in the field of diagnosing celiac disease. More particularly, the present invention relates to a method for diagnosing celiac disease based on the level of anti-gliadin and anti-tissue transglutaminase (tTG) IgA and IgG antibodies.

### BACKGROUND OF THE INVENTION

Celiac disease (CD) is a common small bowel disorder caused by permanent intolerance against gluten proteins in genetically susceptible individuals carrying the HLA-DQ2 (DQA1*0501-DQB1*02) and DQ8 (DQA1*0301-DQB1*0302) haplotypes.¹ Diagnosis is confirmed by intestinal biopsies showing typical histopathological features characterized by flattening of villi, elongation of crypts and increased numbers of intra-epithelial lymphocytes.² Treatment with gluten-free diet leads to healing of the intestinal mucosa structure and reintroduction of gluten results in relapse of the disease.³

Moreover, CD is strongly associated with autoantibodies against tissue transglutaminase (tTG); a calcium-dependent enzyme that belongs to a widely distributed group of enzymes involved in several important physiological processes catalyzing post-translational modification of proteins and peptides.⁴ In CD, tTG is proposed to form new antigens of gluten peptides by deamidation of glutamines to glutamate in gliadin that bind with high affinity to the HLA-DQ2 and DQ8 heterodimers.⁵ Although tTG autoantibodies are highly specific markers for celiac disease, their role in the pathogenesis is still a matter of debate.⁶⁻⁸

Antibodies are useful diagnostic tools in CD. A number of serological tests are already available of which anti-gliadin antibodies (AGA), endomysial autoantibodies (EMA) and tTG autoantibodies of the IgA isotype are commonly measured in clinical practice. The degree of intestinal damage also correlates with antibody levels.⁹ Furthermore, antibodies are reduced after introduction of gluten-free diet, which allows objective evaluation of how patients respond to treatment.¹⁰

In children, the sensitivity and specificity of IgA-AGA has been estimated at 84% (52% to 100%) and 91% (83% to 100%), respectively.¹¹⁻²⁴ The diagnostic performance of IgA-EMA reaches a sensitivity of 96% (88% to 100%) and specificity of 98% (90% to 100%), respectively.^{11, 13-15, 18, 19, 25-29} The expression of IgA-tTG shows a high agreement with IgA-EMA.³⁰⁻³⁵ Using either human recombinant tTG or native human tTG derived from red blood cells as the source of antigen in ELISA kits, the diagnostic sensitivity of IgA-tTG is 96% (86% to 100%) and the specificity is 97% (95% to 100%).^{32, 36, 31} With radioligand binding assays (RBA) for the detection of tTG autoantibodies, the mean sensitivity and specificity reached 98% (96% to 100%) and 99% (96% to 100%), respectively.³⁸⁻⁴¹ As with IgA-EMA, the lower sensitivity of IgA-tTG mainly accounts for the inclusion of children younger than two years of age.⁴²⁻⁴⁵

However, IgA antibodies are insufficient to detect CD in individuals with selective IgA deficiency; a disorder affecting approximately 1/500 of the general population and at a ten-fold increased risk for CD. The estimated sensitivity and specificity of IgG-AGA in children is 93% (83% to 100%) and 82% (65% to 94%), respectively.^{11-14, 19,21, 22, 24} IgG-EMA yields both sensitivity and specificity near 100%, but studies are mainly performed only on patients with IgA deficiency.⁴⁶⁻⁴⁸ On the other hand, the diagnostic validity of IgG-tTG has been shown to be significantly reduced when analyzed by enzyme-linked immunosorbent assays (ELISA).^{9, 49} This contrasts to data on IgG-tTG bound to protein A analyzed by RBA, which display high correlation with IgA-tTGa.^{41, 50}

The reported variation between different tTG autoantibody immunoassays^{25, 41} might be dependent upon how tTG is presented.⁵¹ The ELISA method is most commonly utilized technique for binding tTG antibodies where an excess of the antigen is fixed to the micro-titer plate.³¹ Consequently, only antibodies with the highest binding affinity for the antigen located in close proximity are measured. With the RBA method, or immunoprecipitation assays, antibodies bind to a low amount of radioactive antigen in a solution where all conformational epitopes are available, which render it possible to detect both high as well as low affinity tTG antibodies.³⁹

Recently, antibodies against synthetic deamidated gliadin peptides (DGP) have proven to be more disease-specific and display higher binding affinity than common AGA immunoassays.^{52, 53, 54} These selectively deamidated, fully synthetic peptide based assays achieve a high level of diagnostic performance due to the incorporation of several conformationally intact B cell epitopes derived from the whole native gliadin molecule. However, detecting anti-gliaden antibodies alone does not provide a definitive diagnosis of CD.

The present invention is a method for diagnosing CD by detecting either IgA or IgG autoantibodies to DGP or tTG antigens.

### SUMMARY OF THE INVENTION

The present invention relates to a method for diagnosing celiac disease in a subject by determining if the subject has IgA or IgG autoantibodies to either DGP or tTG antigens. The method includes the steps of: preparing a deamidated gliadin peptide (DGP) antigen and a tissue transglutaminase (tTG) antigen; contacting the DGP antigen and the tTG antigen with a biological sample from the subject to form DGP antigen- and tTG antigen-antibody complexes; and detecting IgA and IgG antibodies in the DGP antigen- and tTG antigen-antibody complexes. Detection of either IgA or IgG antibodies is indicative of celiac disease.

The DGP antigen and the tTG antigen may be mixed together prior to contacting with the biological sample. Alternatively, two separate reactions can be conducted by contacting the DGP antigen with one aliquot of the biological sample and contacting the tTG antigen with another aliquot of the biological sample.

Detecting IgA and IgG antibodies may also include contacting the antigen-antibody complexes with at least one labeled compound that binds to IgA antibodies, IgG antibodies, or both IgA and IgG antibodies. This labeled compound may be Protein A. In other embodiments, the labeled compound may be an anti-immunoglobulin.

A single labeled compound may be used so long as it binds to both IgG and IgA antibodies. Preferably, it does not bind to IgM antibodies. Alternatively, a mixture of two or more labeled compounds can be used, at least one of which binds to IgG and another of which binds to IgA. If the subject is a human, the labeled compound or compounds may be a labeled anti-immunoglobulin derived from a non-human mammal.

The label used to detect the presence of IgA and/or IgM in the antigen-antibody complexes can be direct, such as a florophore that is inherently detectable, or it may be indirect, such as an enzyme that catalyzes the formation of a detectable product from an undetectable substrate. Accordingly, the label may be, for example, an enzyme, a radioisotope, and a fluorescent moiety. In one embodiment, the label is the enzyme, horseradish peroxidase.

The tTG may be derived from any source, but is conveniently obtained from human erythrocytes.

The format of the assay method may be any of a variety of commonly known immunoassays, such as enzyme linked immunoabsorbent assay (ELISA), fluorescent immunosorbent assay (FIA), chemical linked immunosorbent assay (CLIA), radioimmuno assay (RIA), immunoblotting, gel diffusion precipitation reactions, immunodiffusion assays, in situ immunoassays, Western blots, precipitation reactions, agglutination assays, complement fixation assays, immunofluorescence assays, protein A assays, and immunoelectrophoresis assays.

It should be understood that the assay method of the present invention when performed in any of the aforementioned formats can be performed as a single antigen-antibody reaction, or as multiple antigen-antibody reactions each conducted in a separate assay compartment or at different locations on a solid phase, each of which is designed to detect one of the four combinations of antigens and antibodies as follows: anti-DGP IgA autoantibodies bound to DGP, anti-tTG IgA autoantibodies bound to tTG, anti-DGP IgG autoantibodies bound to DGP, and anti-tTG IgG autoantibodies bound to tTG. When performed as a single antigen-antibody reaction, the antigen target is a mixture of DGP and tTG antigens, and the labeled compound or mixture of two or more compounds is/are capable of detecting both IgA and IgG.

In one exemplary embodiment, the assay method is performed in an enzyme linked immunoabsorbent assay system.

Other aspects of the invention are described throughout the specification.

### BRIEF DESCRIPTION OF THE DRAWINGS

FIG. 1 depicts: Antibody levels in children with (A) untreated celiac disease (n=116), (B) treated celiac disease (n=87), in (C) disease controls (n=57) and (D) adult blood donors (n=398) measured with six different ELISA kits: IgAG-DGP/tTG (I); IgAG-DGP (II); IgA-DGP (III); IgG-DGP (IV); IgA-tTG (V); and IgG-tTG (VI). The dotted horizontal lines denote the cut-off level of upper normal.

FIG. 2 depicts: Mean antibody levels in children with celiac disease (n=20) at diagnosis and at three and six months of gluten-free diet. * is p-value<0.05; ** is p<0.001.

### DETAILED DESCRIPTION OF THE INVENTION

The present invention relates to a method for diagnosing celiac disease in a subject by determining if the subject has IgA or IgG autoantibodies to either DGP or tTG antigens. The method can employ a single reaction using a mixed antigen target and a single labeled compound capable of detecting complex formation between the IgA or IgG autoantibodies present in the biological sample from a subject and the antigens. In most instances, this single labeled compound is a protein such as an immunoglobulin that specifically binds to both IgA and IgG antibodies. Alternatively, a mixture of two or more labeled proteins may be used, such as one that binds to IgA and one that binds to IgG.

Accordingly, the diagnostic assay of the present invention can be formatted such that the detection of each of the four types of autoantibodies (i.e. IgA anti-tTG, IgA anti-DGM, IgG anti-tTG and IgG anti-DGM) is performed in four separate assay reactions, in two separate assay reactions, or in a single assay reaction, with the detection of any or all of the four types of autoantibodies being diagnostic for celiac disease. Such a dual antigen/dual antibody approach to diagnosing celiac disease significantly reduces the number of false negatives observed when assays detect less than all four of these types of autoantibodies.

Accordingly, a single assay reaction may be performed using a mixture of the two antigens as a “target”, contacting the target with a biological sample from a subject, and then using a labeled anti-immunoglobulin antibody that can detect both IgG and IgA antibodies that bind to the target, or using a mixture of two separate labeled anti-immunoglobulin antibodies, one that is specific for IgG and one that is specific for IgA. Using this mixed target and dual-binding labeled antibody or mixed labeled antibody, a single reaction can be carried out to detect the presence of any or all of the four types of autoantibodies. In an alternate embodiment, other labeled proteins such as Protein A can be used to detect either IgG or IgA, since Protein A binds to both IgG and IgA antibodies. In yet another embodiment, the labeled compound is chosen specifically not to bind to IgM, because IgM antibodies in a biological sample from a subject that bind to either DGP or tTG are not as diagnostically significant as either IgG and IgA antibodies. For this reason, detecting IgM antibodies that bind to the target antigens could result in false positive results.

In the description that follows, a number of terms used in the field of molecular biology, immunology and medicine are extensively utilized. In order to provide a clearer and consistent understanding of the specification and claims, including the scope to be given such terms, the following non-limiting definitions are provided.

The term “IgAG-DGP/tTG assay system” encompasses a single assay reaction that is, or multiple assay reactions that are, capable of detecting the presence of any or all of the following four types of autoantibodies—IgA anti-DGP, IgA anti-tTG, IgG anti DGP and IgG anti tTG. Detection of any or all of such autoantibodies is considered to be a positive result, i.e. the subject is positive for celiac disease. The method can also be applied to monitor the status of a subject having celiac disease during treatment.

When the terms “one,” “a,” or “an” are used in this disclosure, they mean “at least one” or “one or more,” unless otherwise indicated.

The term “antibody” refers to a molecule which is capable of binding an epitope or antigenic determinant. The term “antibody” includes whole antibodies and antigen-binding fragments thereof, including single-chain antibodies. Such antibodies include human antigen binding antibody and antibody fragments, including, but not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. The antibodies may be from any animal origin including birds and mammals, e.g., human, murine, rabbit, goat, guinea pig, camel, horse and the like.

The term “antigen” refers to a molecule capable of being bound by an antibody or a T cell receptor (TCR) if presented by MHC molecules. The terms “antigen” and “epitope” are interchangeable. An antigen may be additionally capable of being recognized by the immune system and/or being capable of inducing a humoral immune response and/or cellular immune response leading to the activation of B- and/or T-lymphocytes. An antigen may have one or more epitopes (B- and T-epitopes). Antigens as used herein may also be mixtures of several individual antigens.

As used herein, the term “antigen” also refers to either a separate tTG antigen and a separate DGP antigen, or it may refer to a mixture of two antigen species, which are collectively referred to as the “antigen”. In addition, a single “antigen” species, such as a recombinant fusion protein, may include epitopes derived from both tTG and DGP.

The term “antigenic determinant” refers to a portion of an antigen that is specifically recognized by either B- or T-lymphocytes. Antigenic determinants or epitopes are those parts of an antigen that are recognized by antibodies, or in the context of an MHC, by T-cell receptors. An antigenic determinant contains one or more epitopes.

The term “autoantigen” refers to a constituent of self that binds an autoantibody or that induces a cellular response.

The term “autoantibody” refers to an immunoglobulin, antigen specific B cell surface receptor (surface immunoglobulin), or antigen specific T cell receptor directed against self-protein, carbohydrate or nucleic acid.

The term “epitope” refers to a portion of an antigen that is recognized by the immune system, specifically by an antibody (e.g., an autoantibody), B-cell, or T cell, and thus the particular domain, region or molecular structure to which the antibody, B-cell or T-cell binds. An antigen may consist of numerous epitopes while a hapten, typically, may possess few epitopes.

The term “native protein” refers to a protein that contains only those amino acids found in the protein as it occurs in nature. A native protein may be produced by recombinant means or may be isolated from a naturally occurring source.

The term “portion” when in reference to a protein refers to fragments of that protein. The fragments may range in size from two amino acid residues to the entire amino acid sequence minus one amino acid.

The term “subject” refers to an animal, including, but limited to, an ave, ovine, bovine, ruminant, lagomorph, porcine, equine, canine, feline, rodent or primate, for example a human. Typically, the terms “subject” and “patient” are used interchangeably herein in reference to a mammalian subject, particularly a human subject.

The term “sample” is used in its broadest sense. In one sense, it is meant to include a specimen or culture obtained from any source, as well as biological and environmental samples. Biological samples may be obtained from animals (including humans) and refers to a biological material or compositions found therein, including, but not limited to, bone marrow, blood, serum, platelet, plasma, interstitial fluid, urine, cerebrospinal fluid, nucleic acid, DNA, tissue, and purified or filtered forms thereof. Environmental samples include environmental material such as surface matter, soil, water, crystals and industrial samples. Such examples are not however to be construed as limiting the sample types applicable to the present invention.

The term “serum sample” refers to a biological sample comprising serum. It is understood that a serum sample for use in the present methods may contain other components, in particular blood components. Thus, whole blood samples, or blood samples which have been only partially fractionated or separated but which still contain serum, are considered “serum samples” for purposes of the present invention. One skilled in the art can readily obtain serum samples, for example by using conventional blood drawing techniques. Furthermore, the presence of preservative, anticoagulants or other chemicals in the serum sample should not interfere the detection of IgAG-DGP/tTG-specific autoantibodies.

The term “control” or “control sample” refers to one or more sample, such as a serum sample, taken from at least one healthy blood donor. It is understood that when the control comprises multiple samples, the IgAG-DGP/tTG-specific autoantibody level can be expressed as the arithmetic mean, median, mode or other suitable statistical measure of the IgAG-DGP/tTG-specific antibody level measured in each sample. Multiple control samples can also be pooled, and IgAG-DGP/tTG-specific antibody level of the pooled samples can be determined and compared to the subject's sample.

### DGP/tTG Antigen(s)

A variety of DGP and tTG proteins, polypeptides (e.g., synthetic peptides), and chemical analogs are suitable for use in the present invention as antigens for the detection of DGP-specific and tTG-specific autoantibodies. In one aspect, the antigen is a DGP and tTG protein from any species, including, but limited to, an ave, ovine, bovine, ruminant, porcine, equine, canine, feline, rodent or primate, for example a human. The DGP is a synthetic peptide and tTG protein may be in a native form purified from natural sources or in a recombinant form produced using protein engineering technologies, which may have different post-translation modification from the native protein. In the present invention, the synthetic DGP and native or synthetic tTG proteins are produced using standard molecular biology protocols well-known to those skilled in the art.

The antigen according to the present invention may consist of a polypeptide containing DGP and/or tTG in its entirety, or antigenic fragments thereof. Accordingly, the phrase “an antigen from DGP and/or tTG” intends that the antigen binds one or more antibodies that are selective for domains present in DGP and/or tTG. Thus, the antigen may be a polypeptide consisting of DGP (or a fragment thereof), alone or in combination with a polypeptide consisting of tTG (or a fragment thereof). Additionally, the antigen may be a multimer of the DGP (such as a dimer, trimer, etc.), in combination with an antigen consisting of tTG or multimer, with the repeating units separated by non-interfering linking regions such as polyglycine and other small nonpolar amino acids. Such linking regions may or may not include the naturally existing flanking sequences of the epitope.

The native gliadin present in wheat and several other cereals within the grass genus Triticum has an apparent molecular weight of approximately 50 kDa⁶¹. The protein is separated into α, β and γ-gliadins of approximately 266 amino acid residues⁶², 296 amino acid residues⁶³ and 277 amino acid residues⁶⁴ respectively for Triticum aestivum which will vary depending on the cultivar. The enzymatic reaction of tTG with gliadin results in deamination of some of the abundant glutamines in the glycoprotein to glutamic acid. Deamination at positions 140, 148 and 150 of γ-gliadin peptide from residues 134-15365 and a single deamination of glutamine at position 65 of α-gliadin⁶⁶ results in gliadin peptides that are strong activators of T-cells from CD patients. Tripeptide and hexapeptide within these sequences have been observed to increased CD serum antibody binding⁶⁹. Consequently, sequences containing these, multiples of these and other sequences that bind anti-transglutaminase IgG and IgA antibodies may be synthesized or isolated from the native protein for use in the present invention.

The protein tTG is an enzyme (EC 2.3.2.13) of the transglutaminase family that is linked to CD. Anti-transglutaminase antibodies result from gluten sensitivity as a response to Triticea glutens ingestion stimulating transglutaminase specific B-cell response that eventually result in the production of anti-transglutaminase IgA and IgG antibodies⁶⁷. The three dimension structure of this protein is known⁷⁰ and at present, two isoforms of the human enzyme are known, a and b⁶⁸ having 687 amino acid residues and 548 amino acid residues respectively. Based on this information peptides of tTG can be identified and synthesized or isolated from the native protein that bind anti-transglutaminase IgG and IgA antibodies for use in the present invention.

Unless otherwise indicated, the terms “DGP” and “tTG” refers both to native DGP and tTG proteins, as well as variants thereof. As used herein, DGP and tTG variants are DGP and tTG proteins which comprises an amino acid sequence having one or more amino acid substitutions, deletions, and/or additions (such as internal additions and/or DGP and tTG fusion proteins) as compared to the amino acid sequence of a native DGP and tTG proteins, but which nonetheless retain DGP and tTG immunological activity. Such functionally or immunologically equivalent variants may occur as natural biological variations (e.g., polypeptide allelic variants, polypeptide orthologs, and polypeptide splice variants), or they may be prepared using known and standard techniques for example by chemical synthesis or modification, mutagenesis, e.g., site-directed or random mutagenesis, etc. Thus, for example, an amino acid may be replaced by another which preserves the physicochemical character of the DGP and tTG proteins or its epitope(s), e.g. in terms of charge density, hydrophilicity/hydrophobicity, size and configuration and hence preserve the immunological structure. “Addition” variants may include N- or C-terminal fusions as well as intrasequence insertion of single or multiple amino acids. Deletions may be intrasequence or may be truncations from the N- or C-termini.

The variants may have from 1 to 3, to 5, to 10, to 15, to 20, to 25, to 50, to 75, or to 100, or more than 100 amino acid substitutions, insertions, additions and/or deletions, wherein the substitutions may be conservative, or non-conservative, or a combination thereof. Additionally, the DGP and tTG proteins of the present invention may comprise at least 10, at least 12, at least 15, at least 20, at least 25, at least 30, at least 35, at least 40, or at least 50 consecutive amino acid residues of a native DGP protein. Such a variant is preferably at least about 50%, at least about 60%, at least about 70%, at least about 80%, as lest about 90%, or at least about 95% identical to a native DGP and tTG proteins. Furthermore, the DGP and tTG variants may remain immunologically active with an activity of over about 1%, over about 10%, over about 25%, over about 50%, over about 60%, over about 70%, over about 80%, over about 90%, over about 95%, or over about 100% of the immunological activity of the native protein.

Conservative modifications to the amino acid sequence of a DGP and tTG proteins generally produce a polypeptide having functional and chemical characteristics similar to those of the original DGP and tTG proteins. In contrast, substantial modifications in the functional and/or chemical characteristics of a DGP and tTG proteins may be accomplished by selecting substitutions in the amino acid sequence of the DGP and tTG proteins that differ significantly in their effects on maintaining (a) the structure (secondary, tertiary, and/or quaternary) in the area of the substitution or (b) the charge or hydrophobicity of the molecule at the target site. Amino acid sequence modifications can be accomplished by chemical and biological peptide and protein synthetic methods that are well know in the art.

Desired amino acid substitutions (whether conservative or non-conservative) can be determined by those skilled in the art at the time such substitutions are required. For example, amino acid substitutions can be used to identify important residues, to modulate the biological activity of a DGP and tTG proteins, e.g., the binding interactions with a DGP-specific and tTG-specific antibodies, or to decrease unwanted non-specific binding interactions with other molecules in a sample. Suitable amino acid substitutions include, but are not limited to, substituting Ala with Val, Leu, or Ile; substituting Arg with Lys, Gln, or Asn; substituting Asn with Gln; substituting Asp with Glu; substituting Cys with Ser or Ala; substituting Gln with Asn; substituting Glu with Asp; substituting H is with Asn, Gln, Lys, or Arg; substituting Ile with Leu, Val, Met, Ala, Phe, or Norleucine; substituting Leu with Norleucine, Ile, Val, Met, Ala, or Phe; substituting Lys with Arg, 1,4-diamino-butyric acid, Gln, or Asn; substituting Met with Leu, Phe, or Ile; substituting Phe with Leu, Val, Ile, Ala, or Tyr; substituting Pro with Ala; substituting Ser with Thr, Ala, or Cys; substituting Thr with Ser; substituting Trp with Tyr or Phe; substituting Tyr with Trp, Phe, Thr, or Ser; and substituting Val with Ile, Met, Leu, Phe, Ala, or Norleucine. The selection of an amino acid for replacement can also be guided by its hydropathic index and/or hydrophilicity.

In addition, the polypeptide may be fused to a homologous polypeptide to form a homodimer or to a heterologous polypeptide to form a heterodimer. Heterologous polypeptides include, but are not limited to: an epitope to allow for the detection and/or isolation of a DGP and/or tTG fusion polypeptide, such as, polyhistine at either C- or N-terminal to ease the purification; an enzyme or portion thereof which is catalytically active; a polypeptide which promotes oligomerization, such as a leucine zipper domain; and a polypeptide which increases stability, such as an immunoglobulin constant region.

Fusions can be made either at the amino-terminus or at the carboxyl-terminus of a DGP and/or tTG polypeptide. Fusions may be direct with no linker or adapter molecule or may be through a linker or adapter molecule. A linker or adapter molecule may be one or more amino acid residues, typically from about 20 to about 50 amino acid residues. A linker or adapter molecule may also be designed with a cleavage site for a protease to allow for the separation of the fused moieties. It will be appreciated that once constructed, the fusion polypeptides can further be derivatives according to the methods described herein.

The DGP and tTG proteins of the present invention may also be DGP and tTG derivatives, which is a chemically or biologically modified protein, including protein post-translation modification, such as acylation (i.e., acetylation or formylation), biotinylation, carboxylation, deamination, glutathionylation, glycosylation, lipidation (i.e., farnesylation, geranylgeranylation, prenylation, myristoylation, palmitoylation, or stearoylation), methylation, phosphorylation, sulphation, fucosylation, and ubiquitination. Unless otherwise indicated, the term “DGP protein” and “tTG protein” refers both to native proteins, and variants and derivatives thereof. A protein derivative may be modified in a manner that is different in the type, number, or location of the post-translation modification groups naturally attached to the polypeptide. For example, a derivative may have the number and/or type of glycosylation altered compared to the native protein. The resulting derivative may comprise a greater or a lesser number of N-linked glycosylation sites than the native protein.

The DGP and/or tTG polypeptide may also be modified by the covalent attachment of one or more polymers. Typically, the polymer selected is water-soluble so that the protein to which it is attached does not precipitate in an aqueous environment, such as a physiological environment. The polymer may be of any molecular weight and may be branched or unbranched. The polymer each typically has an average molecular weight of between about 1 kDa to about 100 kDa.

Suitable water-soluble polymers or mixtures thereof include, but are not limited to, polyalkylene glycol (such as mono-(C₁-C₁₀) alkoxy-, aryloxy-polyethylene glycol, poly-(N-vinyl pyrrolidone) polyethylene glycol, propylene glycol homopolymers, or polypropylene oxide/ethylene oxide co-polymers), carbohydrate-based polymers (such as dextran or cellulose), polyoxyethylated polyols, and polyvinyl alcohols. Also encompassed by the present invention are bifunctional crosslinking molecules which can be used to prepare covalently attached DGP and tTG polypeptide multimers.

In general, chemical derivatization may be performed under a suitable condition by reacting a protein with an activated polymer molecule. Methods for preparing chemical derivatives of polypeptides will generally comprise the steps of: (a) reacting the polypeptide with the activated polymer molecule (such as a reactive ester or aldehyde derivative of the polymer molecule) under conditions whereby the DGP and tTG proteins become attached to one or more polymer molecules, and (b) obtaining the reaction products. The optimal reaction conditions may vary depending upon the DGP and tTG proteins selected and chemical reagents used, and are generally determined experimentally. The PEGylation of a polypeptide may be carried out using any of the PEGylation reactions known in the art, including, but not limited to, acylation, alkylation, or Michael addition.

### Labeled Compounds

Any labeled compound, usually a labeled protein, that is capable of being detected, either directly or indirectly, and that binds specifically to either or both IgA and/or IgG antibodies is useful in the practice of the present invention. Such labeled compounds are usually labeled proteins, such as Protein A or anti-immunoglobulin antibodies. These labeled proteins are well known in the diagnostic arts.

Labeled anti-IgA, anti-IgG, or a conjugate or mixture of both anti-IgA and anti-IgG antibodies (hereinafter anti-IgAG antibodies, or anti-IgAG Abs) may be used to detect IgA and IgG autoantibodies in the sample that bind to the DGP and/or tTG antigen. To produce the antibodies, human IgG and/or IgA are purified from human serum and injected into an animal such as a rabbit or goat. The animals produce antibodies to the human IgG and/or IgA. The IgA and/or IgG antibodies of the present invention may also be its variants or derivatives as described above.

### Diagnostic Assay

There are many different types of immunoassays suitable for use in the present invention. Any of the well known immunoassays may be adapted to detect the level of DGP and/or tTG-specific autoantibodies in a sample which react with the DGP and tTG antigens, such as, e.g., enzyme linked immunoabsorbent assay (ELISA), fluorescent immunosorbent assay (FIA), chemical linked immunosorbent assay (CLIA), radioimmuno assay (RIA), immunoblotting, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc. For a review of the different immunoassays which may be used, see: The Immunoassay Handbook, David Wild, ed., Stockton Press, New York, 1994. A competitive immunoassay with solid phase separation or an immunometric assay for antibody testing is particularly suitable for use in the present invention. See, The Immunoassay Handbook, chapter 2.

In one exemplary embodiment of the invention, the diagnostic assay is an immunometric assay for detecting the level of DGP and/or tTG-specific autoantibodies in a sample. In the immunometric assay, the DGP and/or tTG antigens are immobilized on a solid support directly or indirectly through a capture agent, such as anti-DGP and/or tTG antibodies (so long as these capture antibodies do not cross-react with the labeled compound). An aliquot of a sample, such as a serum sample, from a subject is added to the solid support and allowed to incubate with the DGP and/or tTG antigens on the solid phase. A secondary labeled antibody that recognizes a constant region in the autoantibodies present in the sample which have reacted with the DGP and/or tTG antigens is added. When the subject is a human, this secondary antibody is an anti-human immunoglobulin. The secondary antibody which is specific for IgA and/or IgG heavy chain constant regions, or combination thereof, may be employed. After separating the solid support from the liquid phase, the support phase is examined for a detectable signal. The presence of the signal on the solid support indicates that autoantibodies to DGP and/or tTG proteins present in the sample have bound to the DGP and/or tTG antigens on the solid support. Increased optical density or radiolabeled signal when compared to the control samples from normal subjects correlates with a diagnosis of CD in a subject.

Solid supports are known to those skilled in the art and include the walls of wells of a reaction tray (e.g., microtiter plates), test tubes, polystyrene beads, magnetic beads, nitrocellulose strips, membranes, microparticles such as latex particles, glass or silicon chips, sheep (or other animal) red blood cells, duracytes and others. Suitable methods for immobilizing proteins and peptides on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional molecule which has the ability to attract and immobilize the capture reagent. The additional molecule can include a charged substance that is oppositely charged with respect to the capture reagent itself or to a charged substance conjugated to the capture reagent. As yet another alternative, the molecule can be any specific binding member which is immobilized upon (attached to) the solid support and which has the ability to immobilize the DGP and/or tTG antigens through a specific binding reaction. The molecule enables the indirect binding of the DGP and/or tTG antigens to a solid support material before the performance of the assay or during the performance of the assay.

The signal producing system is made up of one or more components, at least one of which is a label, which generate a detectable signal that relates to the amount of bound and/or unbound label, i.e., the amount of label bound or unbound to the DGP and/or tTG antigens. The label is a molecule that produces or which may be induced to produce a signal or which causes another component to produce a signal. Examples of labels include fluorescers, enzymes, chemiluminescers, photosensitizers or suspendable particles. The signal is detected and may be measured by detecting enzyme activity, luminescence or light absorbance. Radiolabels may also be used and levels of radioactivity detected and measured using a scintillation counter.

Examples of enzymes which may be used to label the anti-human immunoglobulin include β-D-galactosidase, horseradish peroxidase, alkaline phosphatase, and glucose-6-phosphate dehydrogenase (“G6PDH”). Examples of fluorescers which may be used to label the anti-human immunoglobulin include fluorescein, isothiocyanate, rhodamines, phycoerythrin, phycocyanin, allophycocyanin, o-phthaldehyde, fluorescamine, and Alexa Fluor® dyes (that is, sulfonated courmarin, rhodamine, xanthene, and cyanine dyes). Chemiluminescers include e.g., isoluminol. For example, the anti-human immunoglobulin may be enzyme labeled with either horseradish peroxidase or alkaline phosphatase.

Enzymes may be covalently linked to anti-human immunoglobulin for use in the methods of the present invention using well known methods. There are many well known conjugation methods. For example, alkaline phosphatase and horseradish peroxidase may be conjugated to antibodies using glutaraldehyde. Horseradish peroxidase may also be conjugated using the periodate method. Commercial kits for enzyme conjugating antibodies are widely available. Enzyme conjugated anti-human and anti-mouse immunoglobulin specific antibodies are available from multiple commercial sources.

Biotin labeled antibodies may be used as an alternative to enzyme linked antibodies. In such cases, bound antibody would be detected using commercially available streptavidin-horseradish peroxidase detection systems.

Enzyme labeled antibodies produce different signal sources, depending on the substrate. Signal generation involves the addition of substrate to the reaction mixture. Common peroxidase substrates include ABTS (2,2′-azinobis(ethylbenzothiazoline-6-sulfonate)), OPD (O-phenylenediamine) and TMB (3,3′,5,5′-tetramethylbenzidine). These substrates require the presence of hydrogen peroxide. p-Nitrophenyl phosphate is a commonly used alkaline phosphatase substrate. During an incubation period, the enzyme gradually converts a proportion of the substrate to its end product. At the end of the incubation period, a stopping reagent is added which stops enzyme activity. Signal strength is determined by measuring optical density, usually via spectrophotometer.

Alkaline phosphatase labeled antibodies may also be measured by fluorometry. Thus in the immunoassays of the present invention, the substrate 4-methylumbelliferyl phosphate (4-UMP) may be used. Alkaline phosphatase dephosphorylated 4-UMP to form 4-methylumbelliferone (4-MU), the fluorophore. Incident light is at 365 nm and emitted light is at 448 nm.

The amount of color, fluorescence, luminescence, or radioactivity present in the reaction (depending on the signal producing system used) is proportionate to the amount of autoantibodies in a sample which react with the DGP and/or tTG antigens. Quantification of optical density may be performed using spectrophotometric or fluorometric methods, including flow cytometers. Quantification of radiolabel signal may be performed using scintillation counting.

In another exemplary embodiment, the assay is a competitive immunoassay, which employs one or more DGP and/or tTG-specific antibodies that binds to the same epitopes as the DGP and/or tTG-specific autoantibodies. In the assay, the DGP and tTG-specific antibodies and the DGP and/or tTG-specific autoantibodies in a sample compete for binding to the DGP and/or tTG antigens. Typically, a constant amount of a labeled antibody which is known to bind to DGP and/or tTG antigens is incubated with different concentrations of a sample from a subject. The DGP and/or tTG-specific antibodies may be monoclonal or polyclonal.

As described herein, the anti-immunoglobulin antibodies may be labeled with a fluorescer, enzyme, chemiluminescer, photosensitizer, suspendable particles, or radioisotope. After incubation, bound labeled antibodies are separated from free labeled antibodies. Depending on the signal producing system used and if necessary, an appropriate substrate with which the labeled antibody reacts is added and allowed to incubate. The signal generated by the labeled antibodies is then measured. A decrease in optical density or radioactivity in the presence and absence of the sample or between experimental and control samples, is indicative that autoantibodies in the sample have bound to the DGP and/or tTG antigens. Decreased optical density or radiolabeled signal when compared to control samples from normal subjects correlates with a diagnosis of CD in a subject.

In an alternative exemplary embodiment of the competitive immunoassay, an indirect method using two antibodies is provided. DGP and/or tTG antigen specific antibodies are added first as described in the preceding paragraph with the exception that they are not labeled. They are incubated with different concentrations of a sample from a subject. A constant amount of a second antibody is then added to the mixture of the sample and the first antibody. The second antibody recognizes constant regions of the heavy chains of the first antibody. For example, the second antibody may be an antibody which recognizes constant regions of the heavy chains of mouse immunoglobulin which has reacted with the DGP and/or tTG antigens (anti-mouse immunoglobulin). The second antibody may be labeled with a fluorophore, chemilophore or radioisotope, as described above. Free labeled second antibody is separated from bound antibody. If an enzyme-labeled antibody is used, an appropriate substrate with which the enzyme label reacts is added and allowed to incubate. A decrease in optical density or radioactivity from before and after addition of the serum sample in comparison with control samples is indicative that autoantibodies in the serum sample have bound to the DGP and/or tTG antigens. Decreased optical density or radioactivity when compared to control samples from normal subject correlates with a diagnosis of a CD in a subject.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to autoimmune or chronic inflammatory disease markers is utilized.

In some embodiments, the DGP and/or tTG specific autoantibody level may be used together with other biological markers as a panel for the diagnosis of CD. The panel allows for the simultaneous analysis of multiple markers correlating with CD. For example, a panel may include markers identified as correlating with CD in a subject that is likely or not to respond to a given treatment. Depending on the subject, panels may be analyzed alone or in combination in order to provide the best possible diagnosis and prognosis. Markers for inclusion on a panel are selected by screening for their predictive value using any suitable method, including but not limited to, those described in the illustrative examples below.

### Data Analysis

In the present invention, a computer-based analysis program may also be used to translate the raw data generated by the detection assay into data of predictive value for a clinician. The clinician can readily access the predictive data using any suitable means. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication system). Once received by the profiling service, the sample is processed and a profile is produced, specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of a CD to respond to a specific therapy) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or severity of disease.

### EXAMPLE 1

### Materials and Methods

Serum samples were obtained from 176 children admitted to the Department of Pediatrics, University Hospital MAS in Malmö with suspected CD for the investigation with intestinal biopsy. A total of 119 children (75 females, 44 males) had abnormal biopsies at median 5.7 years of age (range 0.7-19.0) and were diagnosed with CD according to the revised criteria of ESPGHAN (European Society of Pediatric Gastroenterology Hepatology and Nutrition).³ The remaining 57 children (26 females, 31 males) had normal biopsies at median 3.5 years of age (range 0.9-14.6) and were considered to have disorders other than CD. In the disease control group, cow's milk protein intolerance or food allergy were diagnosed in seven children, five had IgA-deficiency, three had lipase deficiency, two had Helicobacter pylori gastritis and three had had transient EMA, one of whom had insulin-dependent diabetes mellitus. Four children were investigated because of failure to thrive, or short stature, and the remaining had transient gastrointestinal symptoms. Also included in the study were 87 children with CD (57 females, 30 males) treated with gluten-free diet for median duration of 4.5 years (range 0.5-16.5). All treated CD children had experienced relief of symptoms and showed clinical signs of remission on gluten-free diet. Additionally, serum samples were taken from 20 children (13 females, 7 males) at diagnosis of CD at median 3.9 years (range 1.3-13.9), and after the first three and six months of gluten-free diet. As healthy controls, serum samples from 398 adult blood donors (136 females, 262 males) at median 44 years of age (range 19-81).

### Intestinal Biopsies

One or several biopsies were taken from the distal part of duodenum by Watson capsule or with upper endoscope. Standard sections stained with hematoxylin and eosin and were examined by a pathologist at the Department of Pathology, University Hospital MAS. Histopathological features were classified according to the Marsh criteria, with slight modifications, and defined as: normal villous and crypt architecture and an intra-epithelial lymphocyte (IEL) count<25/100 enterocytes (grade 0); normal villous and crypt architecture with increased number of IELs; crypt hyperplasia, increased number of IEL and flattening of villi showing partial villous atrophy; subtotal villous atrophy with villous width exceeding length; or total villous atrophy, i.e. flat mucosa.

### Total IgA and EMA

Total serum IgA was determined by turbidimetry⁵⁵ at Clinical Microbiology and Immunology, Lund University Hospital. In samples with concentrations IgA <0.07 g/L, further analysis for establishment of IgA deficiency defined as <0.05 g/L was performed by rocket immunoelectrophoresis.⁵⁶ EMA was detected with fluorescein isothiocyanate conjugated goat anti-human IgA antibodies applied to tissue slides of primate esophagus and visualized by immunofluorescence.⁵⁷ Results were expressed as the highest dilution factor giving a positive fluorescence pattern in microscope. All sera manifesting fluorescence at a titer >1:10 were considered to be positive.

### Tissue Transglutaminase Antibody Radioligand-Binding Assay

Human tTG was synthesized by in vitro transcription and translation as described elsewhere.⁵⁸ One microgram of tTG cDNA, subcloned into the pGEM-T Easy Vector (Promega, Madison, Wis., USA), was used to generate ³⁵S-tTG using the TNT SP6 coupled reticulocyte lysate system (Promega, Madison, Wis., USA) in the presence of 20 μCi³⁵S-methionine (Amersham Pharmacia Biotech, Piscataway, N.J., USA). Efficiency of incorporation (typically 15-20%) of the radioactive label was measured by trichloroacetic acid precipitation of the translational product. Both IgA-tTG and IgG-tTG were analyzed as previously described.⁵⁹ Antibody levels were expressed as relative units (RU) in reference to positive and negative sera: RU=(cpm of sample-mean cpm of two negative controls)/(cpm of positive control-mean cpm of two negative controls)*100. Using a cut-off level representing the 99.9^{th} percentile of 277 healthy control subjects, sera yielding test results >3.5 RU for IgA-tTG and >11.8 RU for IgG-tTG, respectively, were considered as positive.⁵⁹ Borderline values were arbitrarily defined between the 99.0^{th} and 99.9^{th} percentiles and estimated between 2.8-3.5 RU for IgA-tTG and 8.3-11.8 RU for IgG-tTG, respectively. Both the intra- and inter-assay variations were <10%.

### Anti-Gliadin Antibody Enzyme Immunosorbant Linked Assays

Six different ELISAs were tested (INOVA Diagnostics, San Diego, Calif.) and run according to the manufacturer's instructions. Briefly, DGP and/or purified human erythrocyte tTG (htTG) coated ELISA plates were incubated with diluted patient serum samples. Antibodies bound to the ELISA wells were detected with conjugates of horseradish peroxidase labeled anti-human IgA, IgG or IgAG conjugate. Antibody levels were calculated from the optical density of the sample in relation to the reactivity of a positive control and expressed as arbitrary units (AU). Cutoff limits <20 AU were defined as negative, 20-30 AU weakly positive and >30 AU moderate to strongly positive, respectively.

### Statistical Analysis

Differences in antibody levels were tested using the Kruskal-Wallis and Dunn's multiple comparison test. The Wilcoxon signed rank test was used to test significant change in autoantibody levels before and after effect of gluten-free diet. Correlations were evaluated using Spearman rank correlation (r) and p-values<0.05 were considered significant.

### Results

### Untreated CD Children

Among untreated CD children, a total of 32/119 (27%) and 34/119 (29%) had partial villous atrophy and subtotal villous atrophy, respectively, and 48/119 (40%) had flat mucosa. Five of 119 (4%) had biopsy findings corresponding to infiltrative stages with increased numbers of IELs. Five of 119 untreated CD children with biopsies showing partial or total atrophy were EMA negative of whom two had IgA-deficiencies, and the remaining two were younger than three years of age. The other child had transient positive EMA titers while the biopsy showed partial villous atrophy at 4.9 years of age. The analysis of tTG antibodies by RBA, detected IgA-tTG in all but 3/119 (3%) untreated CD children with partial, subtotal and total villous atrophy of whom one was 0.7 years of age and two had IgA-deficiency. IgG-tTG detected 117/119 (98%) including both IgA-deficient children, whereas the two IgG-tTG negative children had a biopsy showing subtotal villous atrophy and infiltrative stages with increased numbers of IELs at 0.9 and 2.7 years of age, respectively.

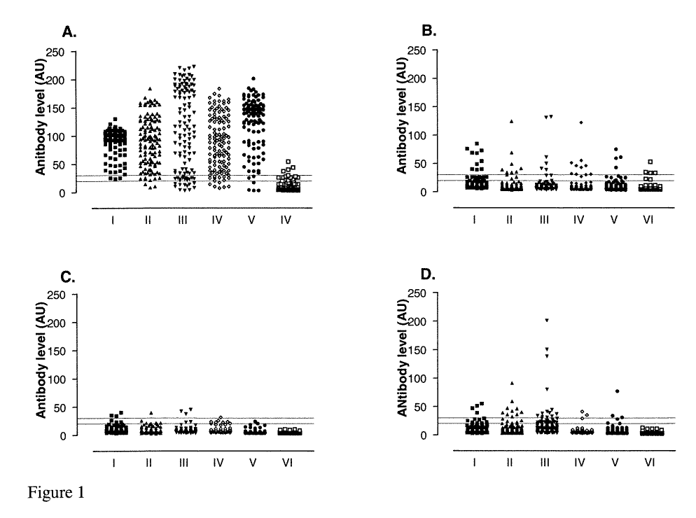

The diagnostic sensitivity and specificity of each ELISA test is summarized in Table 1 and the concordance between a negative and positive test result is shown in Table 2. Using the cut-off level set by the manufacturer to 20 AU for a positive result, the IgAG-DGP/tTG assay detected all of 119 (100%) untreated CD children (FIG. 1, Table 3). Of these children, 116/119 (97%) were positive for IgAG-DGP, 108/119 (91%) for IgA-DGP and 113/119 (95%) for IgG-DGP, respectively. IgA-tTG detected 115/119 (97%), whereas only 15/119 (13%) were positive for IgG-tTG including one of two children with IgA deficiency. The distribution of antibodies revealed that 12/119 (10%) were detected with all six ELISA kits and 91/119 (76%) were positive for all antibodies except for IgG-tTG. The two untreated CD children with IgA-deficiency were negative for both IgA-tTG and IgA-DGP, but positive in the other tests incorporating an anti-IgG conjugate. The outcome of the remaining 14 untreated CD children is shown in Table 3.

TABLE 1
The diagnostic sensitivity and specificity of ELISA tests depending on cut-off level.

| | IgAG-DGP/ | | | | | | | | | | | |
|---|---|---|---|---|---|---|---|---|---|---|---|---|
| ELISA | tTG | | IgAG-DGP | | IgA-DGP | | IgG-DGP | | IgA-tTG | | IgG-tTG | |
| Cut-off 20 AU | | | | | | | | | | | | |
| Sensitivity | 100% | 119/119 | 97% | 116/119 | 91% | 108/119 | 95% | 113/119 | 97% | 115/119 | 13% | 15/119 |
| Specificity | 89% | 51/57 | 89% | 51/57 | 91% | 49/57 | 86% | 49/57 | 96% | 55/57 | 100% | 57/57 |
| (DC) | | | | | | | | | | | | |
| Specificity | 97% | 385/398 | 95% | 380/398 | 92% | 395/398 | 99% | 395/398 | 98% | 392/398 | 100% | 398/398 |
| (BD) | | | | | | | | | | | | |
| Cut-off 30 AU | | | | | | | | | | | | |
| Sensitivity | 97% | 116/119 | 92% | 110/119 | 80% | 105/119 | 88% | 105/119 | 95% | 113/119 | 4% | 5/119 |
| Specificity | 98% | 56/57 | 98% | 56/57 | 95% | 56/57 | 98% | 56/57 | 100% | 57/57 | 100% | 57/57 |
| (DC) | | | | | | | | | | | | |
| Specificity | 99% | 394/398 | 97% | 388/398 | 97% | 396/398 | 99% | 396/398 | 99% | 396/398 | 100% | 398/398 |
| (BD) | | | | | | | | | | | | |

Abbreviation:
BD, blood donors;
DC, disease controls;
DGP, deamidated gliadin peptide;
tTG, tissue transglutaminase.
Cut-off <20 AU denotes weak positive value; >30 moderate to strong positive values. Two children with celiac disease had IgA-deficiency and were negative in IgA-DGP and IgA-tTG.

TABLE 2
The concordance between positive and negative results in untreated CD children and
disease controls (n = 176).

| | | IgA-tTG | IgG-tTG | IgAG- | IgAG- | IgA- | IgG- | IgA- | IgG- |
|---|---|---|---|---|---|---|---|---|---|
| | EMA | (RBA) | (RBA) | DGP/tTG | DGP | DGP | DGP | tTG | tTG |
| EMA | — | 94.3% | 96.0% | 86.4% | 85.2% | 82.4% | 94.3% | 94.9% | 43.8% |
| IgA-tTG | 94.3% | — | 98.9% | 97.7% | 99.4% | 95.5% | 100% | 97.7% | 39.8% |
| (RBA) | | | | | | | | | |
| IgG-tTG | 96.0% | 98.9% | — | 96.6% | 98.3% | 96.6% | 98.9% | 98.9% | 40.9% |
| (RBA) | | | | | | | | | |
| IgAG- | 86.4% | 97.7% | 96.6% | — | 98.3% | 93.2% | 97.7% | 95.5% | 37.5% |
| DGP/tTG | | | | | | | | | |
| IgAG-DGP | 85.2% | 99.4% | 98.3% | 98.3% | — | 94.9% | 99.4% | 97.2% | 39.2% |
| IgA-DGP | 82.4% | 95.5% | 96.6% | 93.2% | 94.9% | — | 95.5% | 97.7% | 44.3% |
| IgG-DGP | 94.3% | 100% | 98.9% | 97.7% | 99.4% | 95.5% | — | 97.7% | 39.8% |
| IgA-tTG | 94.9% | 97.7% | 98.9% | 95.5% | 97.2% | 97.7% | 97.7% | — | 42.0% |
| IgG-tTG | 43.8% | 39.8% | 40.9% | 37.5% | 39.2% | 44.3% | 39.8% | 42.0% | — |

TABLE 3
The distribution of ELISA antibodies in untreated CD children (n = 119).

| | Positive test (+) | | | | | |
|---|---|---|---|---|---|---|
| N patients (%) | IgAG-DGP-tTG | IgAG-DGP | IgA-DGP | IgG-DGP | IgA-tTG | IgG-tTG |
| 91 (76%) | + | + | + | + | + | |
| 12 (10%) | + | + | + | + | + | + |
| 3 (3%) | + | + | | + | + | + |
| 3 (3%) | + | + | + | | + | |
| 2 (2%) | + | | | | + | |
| 2 (2%) | + | + | | + | | |
| 2 (2%) | + | + | | + | + | |
| 2 (2%) | + | + | | + | + | + |
| 1 (1%) | + | + | + | + | | |
| 1 (1%) | + | | + | | + | |

### Disease Controls

Among disease controls one child with cow's milk protein intolerance had had partial villous atrophy and another with Helicobacter pylori gastritis had increased IELs only. The remaining children had normal biopsies. All disease controls were EMA negative although two children had EMA transiently detected before intestinal biopsy at 4.5 and 5.4 years of age, respectively. RBA detected another four disease controls with elevated IgA-tTG levels between 4.1-8.9 RU that had normal biopsies and two of these were also positive for IgG-tTG.

A total of 44/57 (77%) were negative in all ELISA tests whereas 13/57 (23%) children were positive for one antibody or more (Table 4). Six of 57 (11%) disease controls were positive for IgAG-DGP/tTG or IgAG-DGP; all of which had levels lower than 40 AU. IgA-DGP were found in 5/57 (9%) and IgG-DGP in 8/57 (14%) disease controls, respectively, whereas only 2/57 (4%) had IgA-tTG and none IgG-tTG. None of the EMA transient children were considered positive in any of the ELISA tests. On the other hand, in the two children with both IgA-tTG and IgG-tTG detected by RBA one child was positive for IgAG-DGP/tTG (40 AU), IgG-DGP (23 AU) and IgA-tTG (20 AU) and the other child for IgAG-DGP/tTG (35 AU), IgAG-DGP (21 AU) and IgG-DGP (23 AU), respectively. In the two remaining disease controls positive by RBA for IgA-tTG only, one child had also weakly or moderately elevated levels of IgAG-DGP/tTG (34 AU), IgAG-DGP (21 AU) and IgG-DGP (23 AU) and the other IgAG-DGP (21 AU) and IgA-DGP (37 AU), respectively.

TABLE 4
The distribution of ELISA antibodies in disease controls (n = 57).

| | Positive test (+) | | | | | |
|---|---|---|---|---|---|---|
| N patients (%) | IgAG-DGP-tTG | IgAG-DGP | IgA-DGP | IgG-DGP | IgA-tTG | IgG-tTG |
| 44 (77%) | | | | | | |
| 4 (7%) | + | + | | + | | |
| 4 (7%) | | | | + | | |
| 2 (4%) | + | | | + | + | |
| 2 (4%) | | + | + | | | |
| 1 (2%) | | | | + | | |

### Blood Donors

Neither EMA analysis nor intestinal biopsy was performed in blood donors, but RBA detected 2/398 (0.5%) with elevated levels of both IgA-tTG and IgG-tTG and another individual with IgA-tTG only. A total of 353/398 (89%) were negative in all six ELISA kits, while 21/398 (5%) were positive for IgA-DGP only, another 7/398 (2%) for IgAG-DGP and 4/398 (1%) were positive for both antibodies. (Table 5) In addition, 12/398 (3%) were positive for IgA-DGP in combination with one antibody or several other antibodies and one individual was positive for IgG-DGP only. The distribution of antibodies among the blood donors is shown in Table 5.

TABLE 5
The distribution of ELISA antibodies in adult blood donors (n = 398).

| | Positive test (+) | | | | | |
|---|---|---|---|---|---|---|
| N patients (%) | IgAG-DGP-tTG | IgAG-DGP | IgA-DGP | IgG-DGP | IgA-tTG | IgG-tTG |
| 353 (89%) | | | | | | |
| 21 (5.3%) | | | + | | | |
| 7 (1.8%) | | + | | | | |
| 4 (1.0%) | | + | + | | | |
| 2 (0.5%) | + | | | | + | |
| 2 (0.5%) | + | + | + | | | |
| 1 (0.3%) | + | + | + | | + | |
| 1 (0.3%) | + | | + | | + | |
| 1 (0.3%) | + | | | | + | |
| 1 (0.3%) | + | + | | | | |
| 1 (0.3%) | + | + | + | + | | |
| 1 (0.3%) | + | + | | | | |
| 1 (0.3%) | | | | + | | |
| 1 (0.3%) | + | | | | | |
| 1 (0.3%) | + | | + | | | |

In the two blood donors with positive results for both IgA-tTG and IgG-tTG in RBA, one individual was simultaneously positive for IgAG-DGP/tTG (55 AU), IgAG-DGP (22 AU), IgA-DGP (29 AU) and IgA-tTG (77 AU). The other had weakly elevated levels of IgAG-DGP/tTG (25 AU) and IgA-tTG (20 AU) only. The remainder positive for IgA-tTG in RBA had moderately or strongly elevated levels of IgAG-DGP/tTG (51 AU), IgAG-DGP (42 AU), IgG-DGP (41 AU) and IgA-tTG (34 (AU), whereas IgA-DGP was weakly positive (24 AU).

### Treated CD

Despite normal EMA titers, RBA detected elevated levels of both IgA-tTG and IgG-tTG in 10/87 (11%) treated CD children, 9/87 (10%) with IgA-tTG only and another 4/87 (5%) with IgG-tTG, respectively. The duration of gluten-free diet only correlated negatively (r=−0.36) with RBA IgG-tTG levels (p=0.007).

IgAG-DGP/tTG was present in 22/87 (25%) of treated CD children, 11/87 (12%) were positive for IgAG-DGP, IgG-DGP or IgA-tTG, whereas 9/87 (10%) and 6/87 (7%) were positive for IgA-DGP and IgG-tTG, respectively. There was no correlation between antibody levels and duration of gluten-free in any of the ELISA tests, although most antibody positive children were found within the first two years period of gluten-free diet (Table 6). The concordance between positive and negative result was 100% when IgA-tTG (RBA) was compared with IgAG-DGP/tTG, 87% with IgAG-DGP, IgG-DGP and IgA-tTG, 85% with IgA-DGP, and finally, 82% with IgG-tTG.

TABLE 6
Distribution of treated CD children (n = 87) and duration of gluten-free diet (GFD)
intervals.

| Distribution | | | | | | | |
|---|---|---|---|---|---|---|---|
| GFD | | Positive ELISA test | | | | | |
| duration | Patients | IgAG-DGP/tTG | IgAG-DGP | IgA-DGP | IgG-DGP | IgA-tTG | IgG-tTG |
| (years) | N (%) | n/N (%) | n/N (%) | n/N (%) | n/N (%) | n/N (%) | n/N (%) |
| 0.5-2.0 | 13 (15%) | 6 (38%) | 5 (38%) | 2 (15%) | 5 (38%) | 2 (15%) | 2 (15%) |
| 2.1-4.0 | 22 (25%) | 6 (27%) | 1 (5%) | 1 (5%) | 1 (5%) | 4 (18%) | 1 (5%) |
| 4.1-8.0 | 28 (32%) | 5 (18%) | 4 (14%) | 4 (14%) | 3 (11%) | 3 (11%) | 2 (7%) |
| >8.0 | 24 (28%) | 5 (21%) | 1 (4%) | 2 (8%) | 2 (8%) | 2 (8%) | 1 (4%) |

The Effect of a Gluten-Free Diet

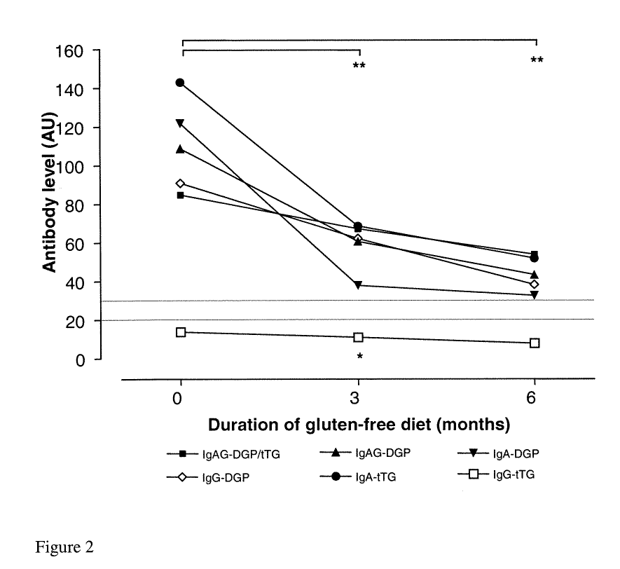

In all twenty children followed from diagnosis there was declined in EMA titers from 1:1600 (median, range 1:100-1:1600) to 1:10 (median, range<1:10-1:400) (p<0.001) after six months of gluten-free diet. Likewise, levels of IgA-tTG (RBA) decreased from 86 RU (median, range 22-131) to 32 RU (median, range 0-99) after six months (p=0.0003) of treatment. Similarly, IgG-tTG levels (RBA) decreased over time from 64 RU (median, range 16-113) to 20 RU (median, range 0-84) at six months (p=0.0002). The antibody response to gluten-free diet is demonstrated in FIG. 2 for all ELISA kits.

### Discussion

This experiment demonstrates the use of an ELISA kit measuring a conjugate of IgA and IgG antibodies against both synthetic gliadin peptides, so called deamidated gliadin (DGP), as well as against human erythrocyte tTG in a pediatric population screened for CD. In addition to this IgAG-DGT/tTG assay, five other ELISA kits were tested; IgAG-DGP, IgA-DGP, IgG-DGP, IgA-tTG and IgG-tTG, respectively. Moreover, these ELISA kits were compared with EMA by indirect immunofluorescence and IgA-tTG and IgG-tTG detected by RBA; two conventional methods for the detection of antibodies with both documented high diagnostic performances. There are several advantages by using conjugated antibodies against both tTG and DGP with the current ELISA compared with analyzing EMA or tTG antibodies alone.

First, it is well known that the diagnostic sensitivity of EMA and tTG antibodies is reduced in very young children, possibly due to an early phase of antibodies directed against gliadin only. As the disease progresses, antibodies are produced against both gliadin and tTG through so-called epitope spreading, which often occurs after two to three years of age. It is therefore recommended to combine AGA with EMA or tTG antibodies in order to increase the diagnostic sensitivity of the test during these age intervals.^{13, 60} However, conventional AGA displays poor specificity to be trusted as a marker to be analyzed alone.¹¹⁻²⁴ As a consequence, young children often undergo invasive endoscopies due to lack of reliable serological markers. However, the new generation of synthetic gliadin peptides has display higher specificity than previous AGA assays in the adult population.⁵²⁻⁵⁴

By using the cut-off level set by the manufacturer for a positive value, 100% of the children with untreated CD were detected by the IgAG-DGP/tTG assay, including those EMA and tTG antibody (RBA) negative children younger than three years of age at diagnosis and those with IgA-deficiency.⁴²⁻⁴⁵ The specificity of this pediatric material was 89%; a lower figure compared with previous reports on the adult population. However, if the cut-off was set at a level defined as moderate to strong positive, the specificity increased to 98% resulting in a decrease in sensitivity to 97%, indicating that the cut-off for might different in the pediatric population. An argument against this is the fact that most disease controls with a positive IgAG-DGP/tTG test were also positive in one or several of other ELISA tests as well. Furthermore, the specificity was calculated from children investigated with gastrointestinal symptoms and it cannot be excluded that other diseases affecting the gastrointestinal tract might moderately elevate the antibody levels. If the specificity was calculated from adult blood donors, the specificity was between 97% and 99%, which is in line with previous results.⁵²⁻⁵⁴

Secondly, there are several methodological benefits by using ELISA compared with the detection of EMA by indirect immunofluorescence or tTG antibodies by RBA. EMA titers are semi-quantitatively established by a subjective operator and therefore not compatible with standardization for large-scale screening. In contrast, RBA objectively assess tTG antibodies with high reproducibility allowing large set of samples to be analyzed with high efficacy at a low cost. However, the use of RBA requires laboratories that can handle radioactive material. The ELISA technique, on the other hand, is simple without handling hazard materials which render it possible for small laboratories to set up the assay. Still, there are some discriminating results that need to be clarified. In this study, the concordance between IgA-tTG results of the two methods was 98%, but only 41% between the IgG-tTG assays. This observation is in line with previous published data where the RBA method showed superior performance of IgG-tTG compared with current ELISA methods.⁵¹

Finally, tTG autoantibodies are reduced following exclusion of gluten from the diet and may therefore also be potential markers for disease activity. From a clinical point of view, this is particularly useful to distinguish children with low from high autoimmunity. For instance, the continuous assessment of autoantibody levels enables the clinician to objectively follow how the child responds to gluten-free diet over time.

### REFERENCES

1. Sollid L M. Molecular basis of celiac disease. Annu Rev Immunol 2000; 18:53-81.
2. Marsh M. Gluten, major histocompability complex and small intestine. Gastroenterology 1992; 102:330-354.
3. Walker-Smith J, et. al., Revised criteria for diagnosis of coeliac disease: Report of Working Group of European Society of Paediatric Gastroenterology and Nutrition (ESPGAN). Arch Dis Child 1990; 65:909-911.
4. Aeschlimann D, et al., Protein crosslinking in assembly and remodelling of extracellular matrices: the role of transglutaminases. Connect Tissue Res 2000; 41:1-27.
5. van de Wal Y, et al., Cutting edge: selective deamidation by tissue transglutaminase strongly enhances gliadin-specific T cell reactivity. J Immunol 1998:1586-1588.
6. Halttunen T, et al., Serum immunoglobulin A from patients with celiac disease inhibits human T84 intestinal crypt epithelial cell differentiation. Gastroenterology 1999; 116:566-72.
7. Esposito C, et al., Anti-tissue transglutaminase antibodies from coeliac patients inhibit transglutaminase activity both in vitro and in situ. Gut 2002; 51:177-81.
8. Dieterich W, et al., Autoantibodies of patients with coeliac disease are insufficient to block tissue transglutaminase activity. Gut 2003; 52:1562-6.
9. Van Meensel B, et al., Diagnostic Accuracy of Ten Second-Generation (Human) Tissue Transglutaminase Antibody Assays in Celiac Disease. Clin Chem 2004; 50:1856-60.
10. Hansson T, et al., Recombinant human tissue transglutaminase for diagnosis and follow-up of childhood coeliac disease. Pediatr Res 2002; 51:700-5.
11. Carroccio A, et al., IgA antiendomysial antibodies on the umbilical cord in diagnosing celiac disease. Sensitivity, specificity, and comparative evaluation with the traditional kit. Scand J Gastroenterol 1996; 31:759-63.
12. Burgin-Wolff A, et al., IgG, IgA and IgE gliadin antibody determinations as screening test for untreated coeliac disease in children, a multicentre study. Eur J Pediatr 1989; 148:496-502.
13. Burgin-Wolff A, et al., Antigliadin and antiendomysium antibody determination for coeliac disease. Arch Dis Child 1991; 66:941-947.
14. Sacchetti L, et al., Diagnostic value of various serum antibodies detected by diverse methods in childhood celiac disease. Clin Chem 1996; 42:1838-42.
15. de Lecea A, et al., Serological screening (antigliadin and antiendomysium antibodies) for non-overt coeliac disease in children of short stature. Acta Paediatr 1996; 412:54-55.
16. Ribes-Koninckx C, et al., A beta-turn rich oats peptide as an antigen in an ELISA method for the screening of coeliac disease in a paediatric population. Eur J Clin Invest 2000; 30:702-8.
17. Grodzinsky E, et al., New automated immunoassay measuring immunoglobulin A antigliadin antibodies for prediction of celiac disease in childhood. Clin Diagn Lab Immunol 2001; 8:564-70.
18. Mascart-Lemone F, et al., Serology of coeliac disease: early diagnosis and therapeutic impact. Acta Gastroenterol Belg 1995; 58:388-96.
19. Lerner A, et al., Immunological diagnosis of childhood coeliac disease: comparison between antigliadin, antireticulin and antiendomysial antibodies. Clin Exp Immunol 1994; 95:78-82.
20. Bode S, et al., The diagnostic value of the gliadin antibody test in celiac disease in children: a prospective study. J Pediatr Gastroenterol Nutr 1993; 17:260-4.
21. Not T, et al., A new, rapid, noninvasive screening test for celiac disease. J Pediatr 1993; 123:425-7.
22. Rautonen J, et al., Antibodies to gliadin in children with coeliac disease. Acta Paediatr
Scand 1991; 80:1200-6.
23. Vitoria J C, et al., Antibodies to gliadin, endomysium, and tissue transglutaminase for the diagnosis of celiac disease. J Pediatr Gastroenterol Nutr 1999; 29:571-4.
24. Chartrand L J, et al., Effectiveness of antigliadin antibodies as a screening test for celiac disease in children. Cmaj 1997; 157:527-33.
25. Bonamico M, et al., Radioimmunoassay to detect antitransglutaminase autoantibodies is the most sensitive and specific screening method for celiac disease. Am J Gastroenterol 2001; 96: 1536-40.
26. Di Leo M, et al., Serum and salivary antiendomysium antibodies in the screening of coeliac disease. Panminerva Med 1999; 41:68-71.
27. Kolho K L, et al., IgA endomysium antibodies on human umbilical cord: an excellent diagnostic tool for celiac disease in childhood. J Pediatr Gastroenterol Nutr 1997; 24:563-7.
28. Uibo O, et al., Human oesophagus: a convenient antigenic substrate for the determination of anti-endomysium antibodies in the serological diagnosis of coeliac disease. Eur J Gastroenterol Hepatol 1995; 7:37-40.
29. Vitoria J C, et al., Use of serological markers as a screening test in family members of patients with celiac disease. J Pediatr Gastroenterol Nutr 1994; 19:304-9.
30. Dieterich W, et al., Autoantibodies to tissue transglutaminase as predictors of celiac disease. Gastroenterology 1998; 115:1317-1321.
31. Sulkanen S, et al., Tissue transglutaminase autoantibody enzyme-linked immunosorbent assay in detecting celiac disease. Gastroenterology 1998; 115:1322-1328.
32. Hansson T, et al., Antibody reactivity against human and guinea pig tissue transglutaminase in children with celiac disease. J Pediatr Gastroenterol Nutr 2000; 30:379-84.
33. Lock R, et al., IgA anti-tissue transglutaminase as a diagnostic marker for sensitive enteropathy. J Clin Pathol 1999; 52:274-277.
34. Troncone R, et al., IgA antibodies to tissue transglutaminase: an effective diagnostic test for celiac disease. J Pediatr 1999; 134:166-170.
35. Koop I, et al., Detection of autoantibodies against tissue transglutaminase in patients with celiac disease and dermatitis herpetiformis. Am J Gastroenterol 2000; 95:2009-14.
36. Vitoria J C, et al., Antibodies to human tissue transglutaminase for the diagnosis of celiac disease. J Pediatr Gastroenterol Nutr 2001; 33:349-50.
37. Wolters V, et al., Human tissue transglutaminase enzyme linked immunosorbent assay outperforms both the guinea pig based tissue transglutaminase assay and anti-endomysium antibodies when screening for coeliac disease. Eur J Pediatr 2002; 161:284-7.
38. Lampasona V, et al., Tissue transglutaminase and combined screening for coeliac disease and type 1 diabetes associated autoantibodies. Lancet 1998; 352:1192-1193.
39. Seissler J, et al., Antibodies to human recombinant tissue transglutaminase measured by radioligand assay: evidence for high diagnostic sensitivity for celiac disease. Horm Metab Res 1999; 31:375-379.
40. Williams A, et al., Evaluation of a high-throughput second antibody radiobinding assay of measuring IgA antibodies to human tissue transglutaminase. J Immunol Meth 1999; 228:81-85.
41. Bazzigaluppi E, et al., Comparison of tissue transglutaminase-specific antibody assays with established antibody measurements for coeliac disease. J Autoimmun 1999; 12:51-56.
42. Mankai A, et al., Tissue transglutaminase antibodies in celiac disease, comparison of an enzyme linked immunosorbent assay and a dot blot assay. Pathol Biol (Paris) 2005; 53:204-9.
43. Fabiani E, et al., The serum IgA class anti-tissue transglutaminase antibodies in the diagnosis and follow up of coeliac disease. Results of an international multi-centre study. International Working Group on Eu-tTG. Eur J Gastroenterol Hepatol 2001; 13:659-65.
44. Baudon J, et al., Diagnosing celiac disease: a comparison of human tissue transglutaminase antibodies with antigliadin and antiendomysium antibodies. Arch Pediatr Adolesc Med 2004; 158:584-8.
45. Agardh D, et al., Tissue transglutaminase immunoglobulin isotypes in children with untreated and treated celiac disease. J Pediatr Gastroenterol Nutr 2003; 36:77-82.
46. Cataldo F, et al., Prevalence and clinical features of selective immunoglobulin A deficiency in coeliac disease: an Italian multicentre study. Gut 1998; 42:362-365.
47. Kumar V, et al., Celiac disease and immunoglobulin a deficiency: how effective are the serological methods of diagnosis? Clin Diagn Lab Immunol 2002; 9:1295-300.
48. Korponay-Szabo I R, et al., Elevation of IgG antibodies against tissue transglutaminase as a diagnostic tool for coeliac disease in selective IgA deficiency. Gut 2003; 52:1567-71.
49. Sblattero D, et al., Human recombinant tissue transglutaminase ELISA: an innovative diagnostic assay for celiac disease. Am J Gastroenterol 2000; 95:1253-7.
50. Seissler J, et al., Autoantibodies to human tissue transgutaminase identify silent coeliac disease in Type I diabetes. Diabetologia 1999; 42:1440-1.
51. Agardh D, et al., Autoantibodies Against Soluble and Immobilized Human Recombinant Tissue Transglutaminase in Children with Celiac Disease. J Pediatr Gastroenterol Nutr 2005; 41:322-327.
52. Aleanzi M, et al., Celiac disease: antibody recognition against native and selectively deamidated gliadin peptides. Clin Chem 2001; 47:2023-8.
53. Prince H E. Evaluation of the INOVA diagnostics enzyme-linked immunosorbent assay kits for measuring serum immunoglobulin G (IgG) and IgA to deamidated gliadin peptides. Clin Vaccine Immunol 2006; 13:150-1.
54. Sugai E, et al., Accuracy of testing for antibodies to synthetic gliadin-related peptides in celiac disease. Clin Gastroenterol Hepatol 2006; 4:1112-7.
55. Stiehm E R, et al., Serum levels of immune globulins in health and disease: a survey.
Pediatrics 1966; 37:715-27.
56. Laurell C B. Electroimmuno assay. Scand J Clin Lab Invest Suppl 1972; 124:21-37.
57. Chorzelski T P, et al., IgA class endomysium antibodies in dermatitis herpetiformis and coeliac disease. Ann N Y Acad Sci 1983; 420:325-34.
58. Grubin C, et al., A novel radioligand binding assay to determine diagnostic accuracy of isoform-specific glutamic acid decarboxylase antibodies in childhood IDDM. Diabetologia 1994; 37:344-450.
59. Agardh D, et al., Prediction of silent celiac disease at diagnosis of childhood Type 1 diabetes by tissue transglutaminase autoantibodies and HLA. Pediatric Diabetes 2001; 2:58-65.
60. Burgin-Wolff A, et al., Antibodies against human tissue transglutaminase and endomysium in diagnosing and monitoring coeliac disease. Scand J Gastroenterol 2002; 37:685-91.
61. Okita T W, et al., Evolution and heterogeneity of the alpha-/beta-type and gamma-type gliadin DNA sequences, J. Biol Chem 260:8203-13, 1985.
62. Kasarda, D. D. et al. Nucleic acid (cDNA) and amino acid sequences of α-type gliadins from wheat (Triticum aestivum), PNAS 1984; 81:4712-4716.
63. Reeves, C. D. and Okita, T. W. Analyses of alpha/beta-type gliadin genes from diploid and hexaploid wheats, Gene 1987; 52:257-266.
64. Arentz-Hansen E. H. et al., Production of a panel of recombinant gliadins for the characterization of T cell reactivity in coeliac disease, Gut 2000; 46:46-51.
65. Molberg, O. et al. Tissue transglutaminase selectively modifies gliadin peptides that are recognized by gut-derived T cells in celiac disease Nat. Med. 1998, 14:713-717.
66. Anderson R. P. et. al. In vivo antigen challenge in celiac disease identifies a single transglutaminase-modified peptide as the dominant A-gliadin T-cell epitope Nat. Med. 2000, 6:337-342.
67. Griffin M. et al. Transmutases: nature's biological glues, Biochem. J. 2002; 368:377-396.
68. Porzio, O. et al. Missense mutations in the TM2 gene encoding transglutaminase 2 are found in patients with early-onset type 2 diabetes, Hum. Mutat. 2007; 28:1150.
69. Osman A. A., et al., B cell epitopes of gliadin, Clin. Exp. Immunol. 2000; 121:248-254.

The examples set forth above are provided to give those of ordinary skill in the art with a complete disclosure and description of how to make and use the preferred embodiments of the compositions, and are not intended to limit the scope of what the inventors regard as their invention. Modifications of the above-described modes (for carrying out the invention that are obvious to persons of skill in the art) are intended to be within the scope of the following claims. All publications, patents, and patent applications cited in this specification are incorporated herein by reference as if each such publication, patent or patent application were specifically and individually indicated to he incorporated herein by reference.

What is claimed is:

1. A method for diagnosing celiac disease in a subject comprising the steps of:
a. preparing a deamidated gliadin peptide (DGP) antigen and a tissue transglutaminase (tTG) antigen;
b. contacting the DGP antigen and the tTG antigen with a biological sample from the subject to form DGP antigen- and tTG antigen-antibody complexes; and
c. detecting IgA and IgG antibodies in the DGP antigen- and tTG antigen-antibody complexes;
wherein detection of IgA or IgG antibodies is indicative of celiac disease.

2. The method according to claim 1, wherein the DGP antigen and the tTG antigen are mixed together prior to contacting with the biological sample.

3. The method according to claim 1, wherein detecting IgA and IgG antibodies further comprises contacting the antigen-antibody complexes with at least one labeled compound that binds to IgA antibodies, IgG antibodies, or both IgA and IgG antibodies.

4. The method according to claim 3, wherein the at least one labeled compound further comprises Protein A.

5. The method according to claim 3, wherein detecting IgA and IgG antibodies further comprises contacting the antigen-antibody complexes with a mixture of a first labeled compound that binds to IgA antibodies and a second labeled compound that binds to IgG antibodies.

6. The method according to claim 5, wherein the first and second labeled compounds are anti-immunoglobulin antibodies.

7. The method according to claim 6, wherein the subject is a human.

8. The method according to claim 7, wherein the anti-immunoglobulin antibodies are derived from a non-human mammal.

9. The method according to claim 3, wherein the label is an enzyme, a radioisotope, or a fluorescent moiety.

10. The method according to claim 3, wherein the label is horseradish peroxidase.

11. The method according to claim 1, wherein the tTG is derived from human erythrocytes.

12. The method according to claim 1, wherein steps b and c are performed in an enzyme linked immunoabsorbent assay system.
